(19) United States
(12) Patent Application Publication
(10) Pub. No.: US 2025/0049586 A1
(43) Pub. Date: Feb. 13, 2025

(54) ACTUATOR AND ORTHOPEDIC TECHNICAL JOINT DEVICE, AND METHOD FOR CONTROLLING SAME

(71) Applicant: OTTO BOCK HEALTHCARE PRODUCTS GMBH, Wien (AT)

(72) Inventors: Dirk SEIFERT, Wien (AT); Roland AUBERGER, Wien (AT); Anna MEIXNER, Wien (AT)

(21) Appl. No.: 18/718,508

(22) PCT Filed: Dec. 14, 2022
(86) PCT No.: PCT/EP2022/085824

§ 371 Date: Jun. 11, 2024

(30) Foreign Application Priority Data
Dec. 16, 2021 (DE) 10 2021 133 497.7

(51) Int. Cl. A61F 2/64; A61F 2/74; A61F 5/01

(52) U.S. Cl. CPC A61F 2/64; A61F 2/74; A61F 5/0123; A61F 2005/0169; A61F 2005/0179

(57) ABSTRACT

The invention relates to an actuator having a main body and at least one fastening device for fastening the actuator to components of an orthopedic technical device, wherein at least one fastening device is mounted displaceably and elastically on the main body.

The invention relates to an actuator having a main body and fastening devices for fastening the actuator to other components. The hydraulic actuator is designed in particular for use in orthopedic devices. The invention also relates to an orthopedic joint device, in particular in prostheses or orthoses with such a hydraulic actuator, and to a method for controlling same.

Actuators, in particular hydraulic actuators, can be designed as purely passive resistance devices such as hydraulic dampers or as active actuators with a drive. Active hydraulic actuators are capable of exerting forces on connected components, for example in order to cause a relative movement of the components to each other. Passive hydraulic actuators are used to influence a relative movement between two components on the basis of external conditions, for example, in orthopedic joint devices, for damping a flexion movement and/or extension movement.

In orthopedic devices such as orthotic joints or prosthetic joints, it is possible to influence the relative pivoting movements depending on movement states and/or sensor data. To influence the flexion resistance and/or extension resistance, the flow resistance in a connecting line between two hydraulic chambers is changed. This can be effected, for example, by an adjustable throttle. The adjustment can be made purely mechanically on the basis of an angular position. Alternatively, the adjustment is made via an actuator which is activated, deactivated or modulated by a control device, wherein the control device initiates the corresponding actions on the basis of sensor data. Sensors are in particular pressure sensors or force sensors, position sensors, acceleration sensors and/or orientation sensors, for example an inertial sensor or an IMU. Separate sensors are complex to assemble and require adapted software for evaluation. In addition, the hydraulic actuators must be adapted accordingly. The same also applies to active hydraulic actuators in which resistances are changed and the forces to be applied are changed.

The object of the present invention is to provide a system that is cost-effective in terms of production, can be modular and requires little adaptation.

This object is achieved by an actuator having the features of the main claim and by an orthopedic joint device and method for controlling same having the features of the additional independent claims. Advantageous embodiments and further developments of the invention are disclosed in the subclaims, the description and the figures.

In the actuator having a main body and fastening devices for fastening the actuator to further components, provision is made that at least one fastening device is mounted movably and elastically on the main body. By means of the serially elastic connection of at least one of the two fastening devices to the main body, a serially elastic element for the actuator is provided with which the functional properties of the actuator are changed without having to make changes to the control system and/or to any sensor technology that is present. The displaceable and elastic mounting of the fastening device on the main body brings about a force-travel relationship of the fastening device to the main body. The maximum displacement of the fastening device relative to the main body, which in particular forms a housing for a cylinder or the cylinder of a hydraulic actuator or the housing for a spindle drive, a lock or a brake, allows a relative movement of the connecting components, for example of the orthopedic device to each other, without further components of the actuator, e.g. the piston in the cylinder, having to move. In the case of a relative movement of the fastening devices to each other and a mobility of components within the actuator, the ranges of movement add up or compensate each other.

In one embodiment, the actuator is designed as a passive linear actuator, in particular as a hydraulic damper, lock or brake, or alternatively as an active linear actuator, in particular as a hydraulic drive or spindle drive. In an embodiment of the actuator as a hydraulic actuator with a piston, a combination of the movements of the fastening device and of the piston in opposite directions can be carried out in a relative movement of the two fastening devices to each other.

In one embodiment, the fastening device is mounted in or on a receiving device, for example a housing or another bearing device, which can also be displaceable or deformable itself, wherein the fastening device is supported on the receiving device in at least one displacement direction via at least one spring element.

Advantageously, the fastening device is supported in both displacement directions relative to the receiving device or to the main body via at least one spring element, so that both tensile forces and compressive forces cause a displacement of the fastening device relative to the main body. The spring element shortens or lengthens under a correspondingly applied force, wherein the travel can be detected via a travel sensor or angle sensor, and the applied force or the moment resulting therefrom can be calculated simply from the known spring stiffness or, in the case of a nonlinear spring element, can be calculated from known force-displacement law. This parameter can in turn be used to control a throttle device, a brake, a spindle drive or the like. The elastic support in both directions of displacement can be realized by two mutually opposing spring elements in order to achieve a spring action. Alternatively, this can be done by means of a single spring element, which can be loaded in both tension and compression. In particular when using only one spring element, which can also be designed as a spring pack or a combination of several elastic elements, it is pretensioned such that a lower force threshold has to be exceeded in order to bring about a displacement of the fastening device and thus a change in length of the hydraulic actuator as a whole. Pretensioning can also be provided in the case of two mutually opposing spring elements, in order to achieve a defined force threshold or hysteresis of the elastic properties of the actuator. Alternatively or in addition to pretensioning, play can also be provided, as a result of which the force acting via the spring element does not initially substantially change in the event of a change in length around the play.

In one embodiment, the spring element is assigned at least one sensor for measuring the deformation, in particular the elastic deformation or displacement of an end point of the spring or of at least two points in connection with the elastic element. It is thereby possible to dispense with the use of force sensors and to use much simpler, more robust and more cost-effective position sensors or travel sensors.

In one embodiment, the receiving device is fastened releasably to the main body, for example screwed on, plugged on, clipped on, or repeatedly releasably fastened in another way by form-fit and/or force-fit engagement. It is possible to arrange and secure different receiving devices on the main body, resulting in a modular construction of the actuator from the main body and the receiving device. Different receiving devices can be equipped with different components, for example spring elements and/or sensors, such that an adaptation to different conditions of use and/or to different patients can be easily made, without the need for complex adaptation of the components of the actuator, e.g. the hydraulics, the mechanics or the control. In one embodiment, the receiving device with an elastic element can be replaced by a substantially rigid component, and vice versa. In particular, the rigid component can be designed as a sensor, in particular to measure forces and moments. This allows the sensor set to be adapted in order to differentiate functionality and manufacturing costs in different variants, while other components remain the same.

In one embodiment, the spring element or the spring elements is or are mounted exchangeably in the receiving device, thereby allowing a variation of the function of the receiving device and thus of the module of the elastically mounted fastening device. Alternatively or in addition, the spring element or the spring elements is or are assigned an adjustable pretensioning device, by means of which it is possible to adapt to different requirements or patients without exchanging components. The adjustable pretensioning device adjusts the pretensioning of the spring, thus altering the elastic properties and permitting adaptation to different users and preferences or uses. The adjustable pretensioning device adjusts the pretensioning of the spring, thus altering the elastic properties and permitting adaptation to different users and preferences or uses.

In one embodiment, the spring element has a non-linear, in particular progressive spring characteristic curve, whereby the behavior of the fastening device can be adjusted with increasing displacement. Using the progressive spring characteristic curve, it is possible to set the maximum displacement and, in particular, to avoid hard contact against an end bearing or a stop. Irrespective of the spring characteristic curve, it is also possible for end stops to be designed to be adjustable, in order to be able to adapt the behavior of the actuator. Several springs or spring elements can be arranged in series and/or in parallel to one another in order to set or implement the respective non-linear spring characteristic curve. Nonlinearity can be achieved by a nonlinear geometry, for example variable lever arms on elastic elements with increasing deformation or closing and opening contact surfaces. Alternatively or in addition, the spring element or parts thereof can be made of materials with non-linear material properties, for example a hyperelastic material. In addition to spiral springs and disk springs, it is also possible to use solid bodies made of an elastic or hyperelastic material. In the case of layered disk spring packs, it is possible to provide cambered intermediate washers. In particular, with a non-linear spring, it is possible to achieve a variation in stiffness by changing the pretensioning.

Gas springs are also possible in principle and, through variation of the gas volume or gas pressure, offer a wide range of possibilities for adjusting the spring properties.

In addition to the use of resilient elements or spring elements, it is provided in one embodiment that the fastening device is mounted in a damped manner, for example by one or more damping elements, as a result of which a hard stop can be prevented when the maximum change in length is reached or after the maximum displacement travel of the fastening device has been reached. In addition, vibrations can be suppressed by the damped mounting. The damping can also be provided by a fluid, which has a dissipative action upon displacement of the fastening device, in particular in the receiving device. For this purpose, the fastening device can be connected to a piston element which, for example, is mounted in the receiving device and which moves a fluid through a throttle in the piston element or an overflow channel.

In one embodiment, the actuator is designed as a hydraulic linear actuator having a housing in which a cylinder is arranged or formed in which in turn a piston is guided on a piston rod protruding from the main body, which piston rod divides the cylinder into two chambers, which are connected to each other via at least one overflow channel, wherein in the overflow channel there is arranged at least one adjustable throttle device via which the flow resistance is adjustable. In one embodiment, at least one fastening device for fastening the hydraulic actuator is mounted movably and elastically on the piston rod, analogously to the fastening device described above. It is also possible for two fastening devices to be mounted movably and elastically on the main body, the piston rod being regarded as part of the main body. The elastic properties of the entire system can be adjusted by the serial or parallel arrangement of a plurality of fastening devices with elastic elements.

Advantageously, the fastening device is mounted displaceably in at least one displacement direction of the piston of the hydraulic actuator, in order thereby to achieve a combined force effect or displacement upon a relative movement of components that are fastened to or in the hydraulic actuator. In the case of a spindle drive or another linear actuator, the fastening device is mounted displaceably in at least one displacement direction in the force direction of the linear actuator, in order thereby to achieve a combined force effect or displacement upon a relative movement of components that are fastened to or in the actuator.

In one embodiment, the fastening device and/or the piston, in the embodiment as a hydraulic actuator, are or is assigned at least one position sensor with which it is possible to detect the respective position of the fastening device or of the piston within the receiving device or relative to the main body or within the cylinder. From a combination of the positions of the fastening device in conjunction with the known spring characteristic, it is possible to draw conclusions regarding the spring force and the stored energy, as a result of which it is possible to dispense with force sensors.

In an embodiment as a hydraulic actuator, an auxiliary piston is mounted elastically on the piston. The piston and the auxiliary piston are assigned at least one position sensor for detecting their positions or for detecting their position relative to each other, as a result of which it is possible to determine the forces acting on the auxiliary piston, since the spring rate or spring characteristic curve of the elastic element or of the elastic elements is known. The arrangement of the auxiliary piston can be carried out in combination with the elastic mounting of the fastening device or also on its own, so that only the auxiliary piston is mounted elastically on the piston within the hydraulics. By means of the elastic mounting of the auxiliary piston on the piston with a compressible space between the piston and the auxiliary piston, it is possible to integrate the force-storing functions and the force-limiting function in the piston via the elastic mounting. By measuring both piston positions or measuring the distance between the two pistons, it is possible to determine the forces within the hydraulic actuator, especially when the throttle device is closed.

The elastically mounted component, either the fastening device or the auxiliary piston, can be assigned a blocking and enabling device, wherein the blocking and enabling device is switchable in order to block the component in a pretensioned position and enable it at a later time so as to once again release the energy stored in the elastic element. The elastically mounted component is compressed, for example at the end of the swing phase, when the actuator decelerates or is stopped or when a delay is otherwise initiated, for example when the throttle device is closed in a hydraulic actuator, wherein the elastic component prevents an abrupt deceleration of the lower part or the upper part. The stored energy can either be delivered directly to support an extension movement or can initially be conserved by activation of a blocking device. At a later point in time, the blocking device can then be deactivated so that it is in an enabling position and the stored movement energy returns to the joint device.

The blocking device can be provided, for example, as a switchable clamping device, brake wedge, toothing or the like for holding the spring in a tensioned position.

The same mechanism and the same use of the energy can be used in the actuator itself which, in one embodiment, is assigned a blocking and enabling device, wherein the blocking and enabling device is switchable in order to block an elastically deformable component or an elastically deformable device of the actuator in a pretensioned position and enable it again at a later time so as to once again release the energy stored in the elastically deformable component or the elastically deformable device.

In one embodiment, the actuator is coupled to a control device, which is coupled to at least one sensor, wherein the control device is configured to control the actuator on the basis of sensor data, such that the linear actuator is controllable as a function of sensor data.

In the orthopedic joint device, having an upper part, a lower part mounted pivotably thereon about a pivot axis, and an actuator, as described above, which is fastened to the upper part and the lower part and provides a resistance to a pivoting movement, provision is made that the joint device is assigned an angle detection device via which the angle between the upper part and the lower part can be detected. The actuator, which can be designed as both passive and active actuator, is secured to the upper part and the lower part via the fastening devices. By way of the angle detection device of the joint device, in combination with the elastic mounting of a fastening device and in particular the detection of a displacement of the fastening device relative to the main body or the receiving device, it is possible to infer, from path information and angle information, the force which is transmitted or applied via the actuator, which means that conclusions can be drawn regarding the joint moment that acts about the pivot axis, and the values can be used as a basis for the control system.

In one embodiment, the angle detection device is configured as an angle sensor or has at least two spatial position sensors, one of which is arranged on the upper part and another on the lower part. The angle detection device is formed via the knee angle sensor or the two spatial position sensors, wherein the angles or spatial positions can already be used for controlling the actuator. By means of these comparatively simple sensors, which are already used to record the parameters during use of the orthopedic joint device, it is possible to achieve a simple, cost-effective and robust control system without the need for complex retrofitting and comparatively complex force sensors.

The angle detection device can comprise a position sensor for detecting the piston position, the actuator position and/or the deformation of the elastic elements. In the case of a multi-part piston with an elastically mounted auxiliary piston, the position sensor can detect both the position of the piston and also the relative position of one piston with respect to the other piston.

In one embodiment, the force acting on the elastic element is determined, in at least one of the two end positions of the actuator, from the determined total length and/or change in length of actuator and elastic element. The total length can be measured directly, but it can also be determined from one or more coupled degrees of freedom of a mechanism connected to the actuator, for example from the angle between the hingedly connected upper part and lower part. If the actuator is located in one of the two end positions, as can be ascertained for example from the total length of actuator and elastic element, especially at low forces acting on the actuator, then, in the event of a further change in length of the serially connected actuator and elastic element, it can be inferred that the change in length is essentially due to the deformation of the elastic element, and the force can be inferred using the force-displacement law. Even before the end positions of the actuator are reached, a force can be inferred if the total length of actuator and elastic element is greater or smaller than the total length without load in the respective end positions of the actuator. The same applies for a rotary actuator with a serially elastic element and the like. The total length can also be the relative displacement and/or twisting of two fastenings, for example on an upper part and a lower part. This embodiment is particularly advantageous if there is no sensor present that measures the length and/or length change of the serially elastic element or of the elastically mounted component.

In addition or alternatively, the force acting on the elastic element can be determined if the actuator is blocked or has a very high resistance to movement compared to the elastic element. In such a situation, the determined change in length is essentially attributable to the deformation of the elastic element. Correspondingly, the force can be inferred using the force-displacement law of the elastic element. In particular, it can be detected and/or stored in the control system that the actuator is blocked or has a high resistance to movement, in particular if at the same time only a low force is applied and, from this point on, the force can be determined from the relative change in length. If the block is canceled or the resistance to movement is reduced, the calculation can be adjusted accordingly or suspended. In the case of an incomplete block, the force can be estimated on the basis of the force-displacement law of the elastic element. This embodiment is also of particular advantageous if there is no sensor present that measures the length and/or length change of the serially elastic element.

In a method for controlling an orthopedic joint device having an upper part, a lower part mounted pivotably on the latter about a pivot axis, and an actuator, as described above, which is arranged between the upper part and the lower part and has a main body and at least one fastening device for fastening the actuator to the upper part or the lower part, wherein the actuator provides resistance to a pivoting movement of the upper part relative to the lower part, and the resistance is changed as a function of sensor data, provision is made that a component of the actuator is mounted elastically in or on the main body, and the position of a component, in particular of the elastically mounted component, is detected and the resistance of the actuator is changed on the basis of the position data of the component, in particular of the elastically mounted component. In particular, a fastening device by which the actuator is coupled to the upper part or the lower part is to be regarded as an elastically mounted component. Alternatively or in addition, the elastic component is an auxiliary piston that is mounted elastically on a piston, or a piston that is mounted elastically on a piston rod.

By means of the method for controlling the orthopedic joint device with the actuator, in particular a hydraulic actuator, in which or on which an elastically mounted element or an elastically mounted component is arranged in particular in serial connection to the piston rod or the hydraulic actuator, energy storage can be generated during the relative movement of upper part and lower part. For example, during a knee flexion, energy can be stored in the elastic element or the elastic elements in the swing phase, which energy supports the subsequent swing phase extension. In this way, more energy can be made available in the swing phase extension, and it does not have to be applied by the user of the orthopedic joint device. In addition, the duration of the swing phase can be influenced, as a result of which a symmetrical gait pattern can be achieved, in particular at higher walking speeds.

The energy storage is effected by an elastic element arranged serially with respect to the actuator, and also serially active, in combination with the in particular hydraulic resistance device, wherein the hydraulic resistance device is controlled via the adjustable throttle. The control is based on sensor data provided by sensors of a control device. The control device is equipped with the necessary hardware components, in particular a processor, a memory device and a necessary power supply. On the basis of the sensor data, there takes place either an evaluation or directly an activation or deactivation of the throttle or of an actuation device of the throttle, for example a motor or a switch. For example, if the throttle is closed in order to limit a maximum flexion angle, the spring element is compressed on account of the serial arrangement of the elastic mounting, such that kinetic energy is stored as potential energy in the spring element or the elastic bearing. This potential energy is either automatically resupplied to the movement, immediately after reversal of the direction of the joint device, or initially stored. The storage is effected via a blocking device which holds the spring element in the tensioned position and blocks or delays the release of the potential energy. At a later time, the energy can then be resupplied if the blocking device is deactivated accordingly. The energy in the elastically mounted component is also stored and released using other operating principles of the actuator, i.e. also in the case of brakes, locking mechanisms, spindle drives, etc.

The change in resistance is effected in particular on the basis of a detected angle between the upper part and the lower part, for example to limit the maximum flexion or to prevent an unbraked impact on a mechanically predetermined extension stop.

The angle between the upper part and the lower part can be detected by an angle sensor and/or a position sensor for detecting the position data of the elastically mounted component or components.

The elastically mounted component can be integrated as a switchable elastic element in a hydraulics system, for example by the auxiliary piston or by an elastic mounting of the hydraulic piston on the piston rod. Alternatively or in addition, the elastically mounted component is arranged or formed outside the actual hydraulics on a housing component or the piston rod. The elastic mounting ensures gentle braking when the flow resistance increases, for example to limit the flexion angle. By means of the elastic mounting, the requirements in respect of the characteristics and the implementation of a corresponding open-loop or closed-loop control are reduced in terms of the accuracy of the valve positions and the switching times. Likewise, in the event of a sudden angle limitation, the accelerations are reduced by an angle-dependent activation of a lock. This protects the structural components and reduces the peak reaction moments for the user. This increases the ease of use for the respective patient.

The actuator increases the resistance when a dynamically predefined target angle is reached, in order to permit elastic braking of the movement. If energy applied during elastic braking is stored in whole or in part, the stored energy can be returned in whole or in part, in particular in a controlled manner, to support the reversal of movement.

The measuring and control principles described above can in principle also be applied to rotary actuators in combination with rotation springs. In particular, the combination of a rotation spring with a rotary brake or lock is of interest in this context. As an alternative or in addition, a translational actuator with an elastic rotation element can be connected in series, e.g. via a mechanism, or a rotary actuator with a translational elastic element. A combination of several translational and/or rotational elements is also possible.

In one embodiment, the maximum relative displacement within the elastic element is small compared to the maximum relative displacements in the actuator. For example, on account of the elastic element, the pivoting of upper part relative to lower part can be only a few angular degrees, whilst the actuator permits pivoting of 120°. The advantage of such an embodiment is that the serially elastic behavior is not noticeable, or barely noticeable, to the user.

A deformation of an elastic element can lead to a relative change in length and/or to a relative rotation and can be determined by detection of the change in length and/or rotation by at least one sensor. It is also possible that, in the case of a translational displacement of the ends of an elastic element and/or of the fastenings, a rotation and/or tilting of elements within the elastic element, parts of the elastic element and/or of the adjacent components take place, and these are measured by at least one sensor. The operating principle of the at least one sensor can be based on electrical properties of the elastic element, parts thereof and/or surrounding components, in particular on variable resistances, capacitances and/or inductances associated with the deformation. By changing resistances, capacitances and/or inductances, frequencies of a resonant circuit can be changed and this change determined. It is also possible that an electromagnetic field, which changes with the deformation, or individual properties and/or components of the field are determined via sensors. For example, a field strength, a field orientation, a flow, a flow direction and/or an orientation of the flow in one or more spatial directions can be determined, in particular of a magnetic field. In one embodiment, the displacement and/or tilt of one or more magnets relative to one or more sensors can be determined via the sensors. Alternatively or in addition, the relative distance and/or the relative tilt of two components can be determined via electromagnetic radiation and/or sound waves, which are emitted for example via an emitter, interact with one or more components and are received by the one or more sensors. In particular, the deformation can be determined via one or more amplitude changes, frequency shifts, runtime measurements, interference and or triangulations of the electromagnetic radiation and/or sound waves.

It is possible that an elastic element in the mechanical behavior has not only purely elastic components but also non-elastic components, for example static friction, sliding friction, viscous components and/or other dissipative components.

Exemplary embodiments of the invention are explained in more detail below on the basis of the appended figures. In the figures:

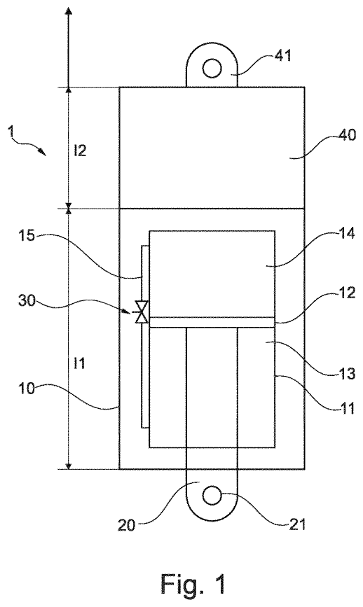

FIG. 1 shows a schematic representation of a hydraulic actuator;

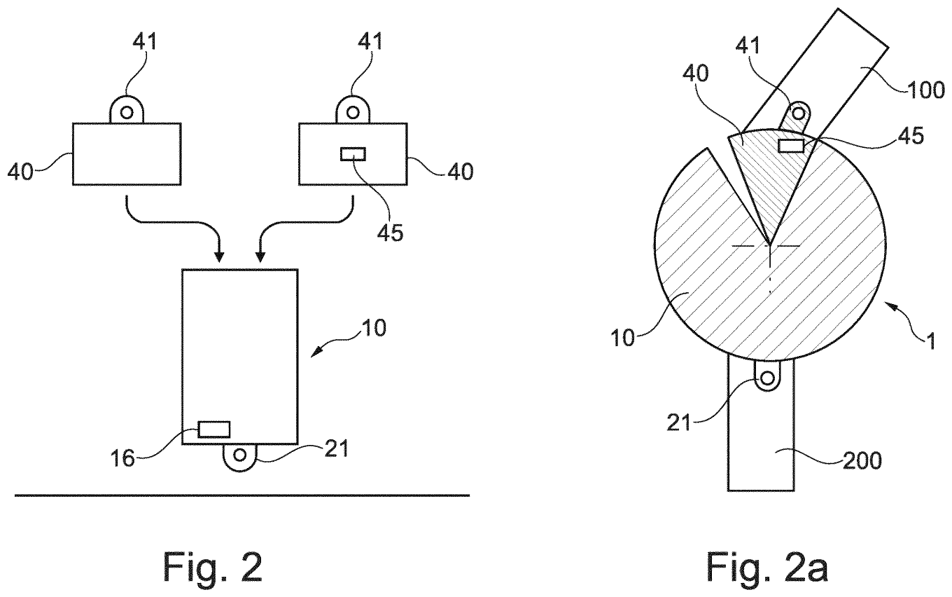

FIG. 2 shows a schematic representation of the modular structure;

FIG. 2a shows a schematic representation of a rotary actuator;

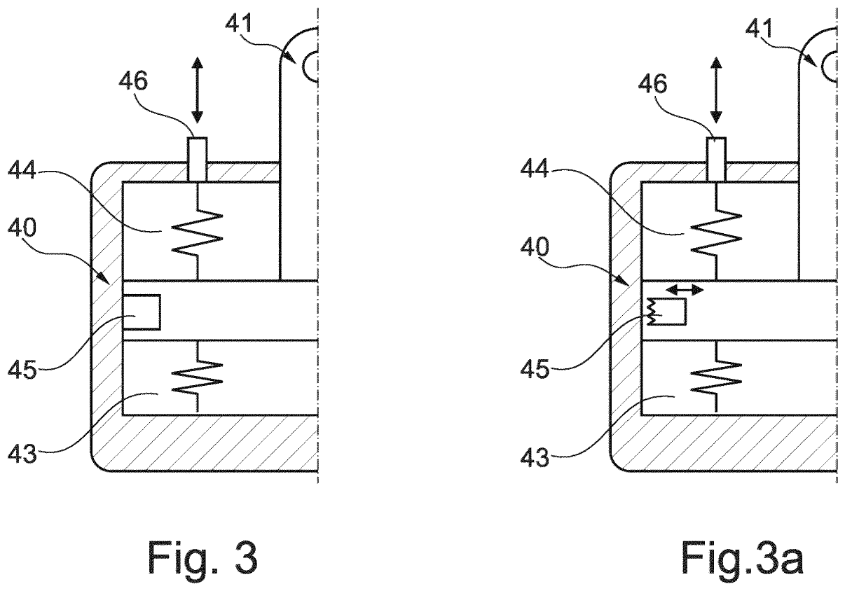

FIG. 3 shows a detailed view of the fastening device;

FIG. 3a shows a variant of the fastening device with blocking device;

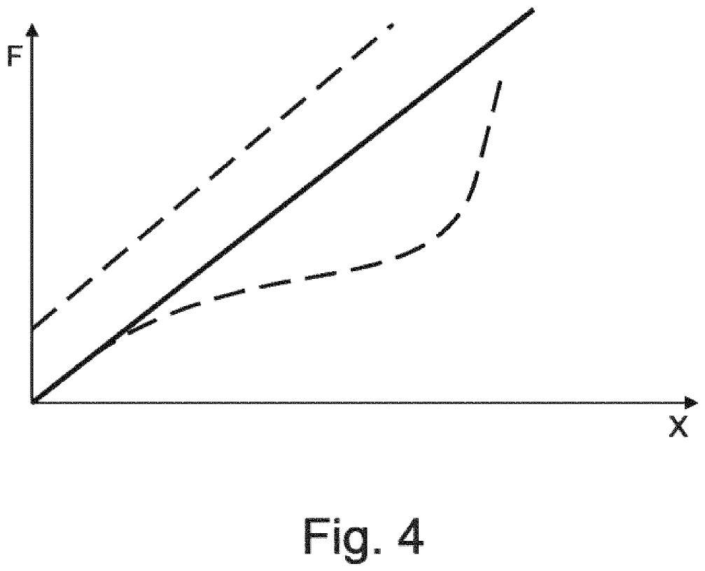

FIG. 4 shows illustrations of spring characteristic curves;

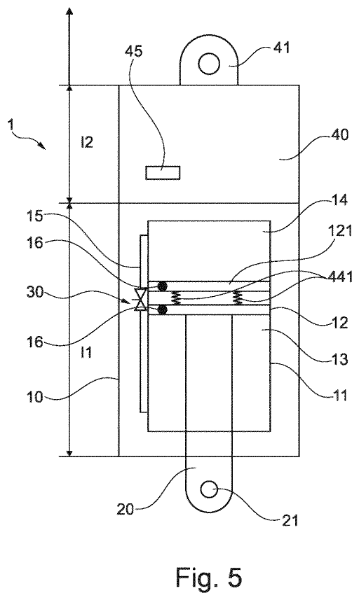

FIG. 5 shows a variant of FIG. 1;

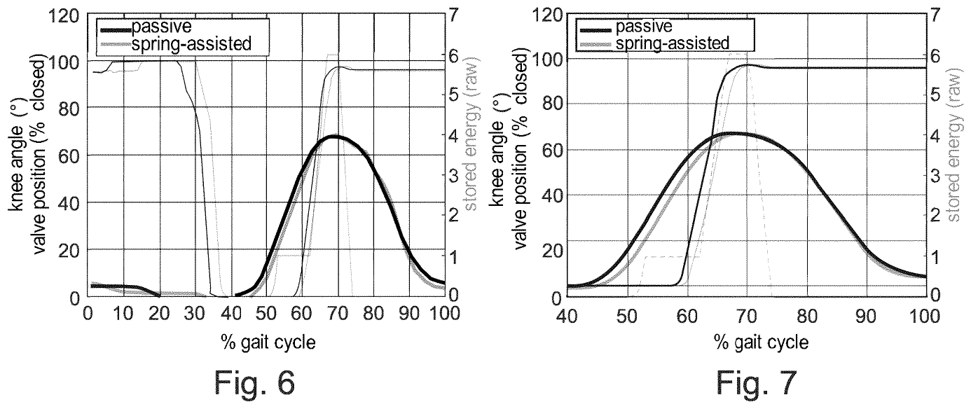

FIGS. 6 and 7 show knee angle curves and positions of flexion valves over the gait cycle;

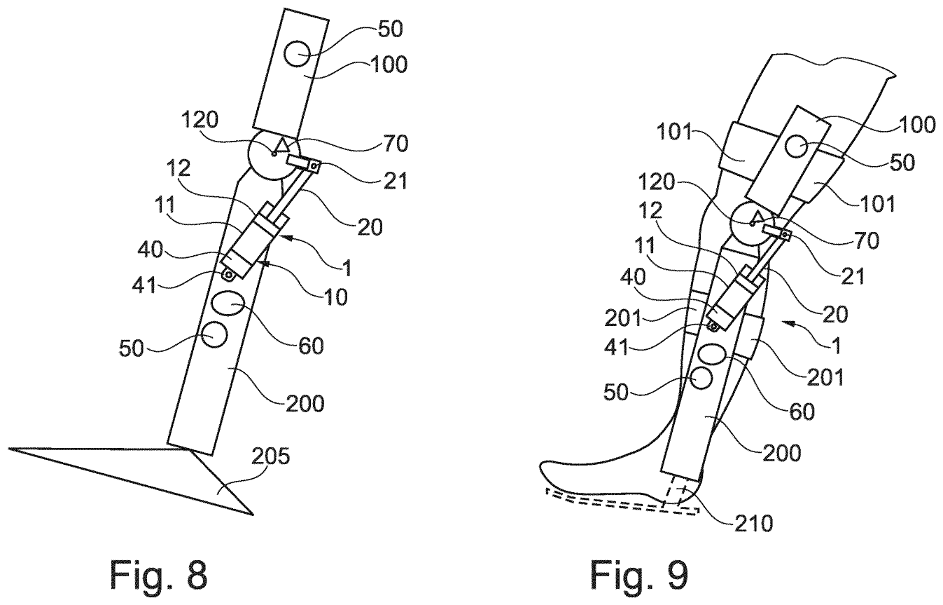

FIG. 8 shows a schematic representation of a prosthetic leg;

FIG. 9 shows a schematic representation of a knee orthosis; and

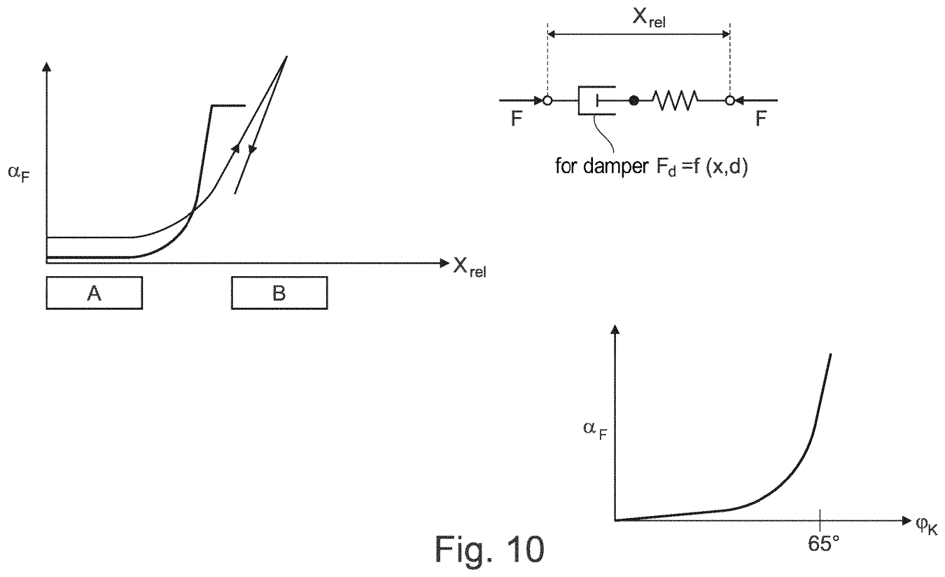

FIG. 10 shows a schematic representation of a spring-damper system.

FIG. 8 illustrates a schematic representation of an artificial knee joint as part of a prosthesis, and FIG. 9 illustrates a schematic representation of an artificial knee joint as part of an orthosis. The artificial knee joint has a upper part 100 and a lower part 200, which are mounted pivotably on each other about a pivot axis 120. In an embodiment as a prosthesis, a prosthetic foot 205 is arranged at the distal end of the lower part 200, while in the embodiment of the artificial knee joint as an orthotic knee joint, as shown in FIG. 9, the lower part 200 is designed as a lower-leg rail, on which no foot part is arranged, but on which an optional foot part 210 may be arranged, as is indicated by the broken line. In the case of a knee-ankle-foot orthosis, a foot part 210 on which a foot can be placed is arranged on the lower part 200. However, this can also be omitted in order to realize what is purely a knee orthosis. In the embodiment as a prosthetic leg according to FIG. 8, a prosthesis socket or another device for receiving a thigh stump or for securing to a person is arranged or formed on the upper part 100. In the embodiment according to FIG. 9, the orthosis is secured to a leg via fastening means 101, 201 which are designed, for example, as straps, shells or the like, in order to secure the orthosis detachably to the leg.

Between the upper part 100 and the lower part 200, an actuator 1 is arranged as a linearly acting hydraulic damper. In the illustrated embodiment, the hydraulic actuator 1 is formed with a hydraulic chamber or a cylinder 11, which is arranged or formed in a housing or main body 10. A piston 12 is mounted movably in the cylinder 11. The piston 12 is displaceable along the longitudinal extent of the cylinder 11 and fastened to a piston rod 20, which protrudes from the housing or main body 10. The piston 12 divides the cylinder 11 into chambers, which are fluidically connected to each other via a hydraulic line, which will be explained later. The main body 10 or the housing can be mounted pivotably on the lower part 200 in order to prevent the piston 12 from jamming during a pivoting movement of the upper part 100 relative to the lower part 200. The end of the piston rod 20 facing away from the piston 12 is fastened to the upper part 100, in the illustrated embodiment on a bracket for increasing the distance to the pivot axis 120. During flexion, the piston 12 is pressed downward, so that the volume of a flexion chamber decreases; correspondingly, the volume of an extension chamber increases, reduced by the volume of the retracting piston rod 20. On account of the flow resistance within the hydraulic line between the extension chamber and the flexion chamber, a resistance is opposed to a flexion movement. The resistance is adjustable. Different volume changes in the extension chamber or flexion chamber are compensated by means of a compensating volume.

In the illustrated embodiment, the actuator 1 is secured to the lower part 200 via a receiving device 40 and a fastening device 41. Both on the upper part 100 and on the lower part 200, a sensor 50 is arranged for detecting the spatial orientation of the lower part 200 and of the upper part 100, respectively. By way of this sensor 50, which can be designed for example as an inertial measurement unit (IMU), the solid angle or the absolute angle to a fixed spatial orientation, for example the gravitational direction, is determined during the use of the artificial knee joint. Instead of an IMU, the sensor 50 can also record other status data, in particular status data which affect the artificial knee joint. In particular, positions, angular positions, velocities, accelerations, forces and their profiles or changes are recorded as status data. The determined solid angle of the upper part 100 and/or of the lower part 200 or another status variable is compared against a threshold angle. When a threshold value is reached or exceeded, which is stored in a controller for the respective sensor value or a value derived from it, an actuator is activated or deactivated in order to change the flow resistance in the actuator 1.

The actuator 1 in an artificial knee joint serves to moderate a flexion movement and an extension movement in order to generate or support an appropriate or desired sequence of movement. An extension movement may be supported and is advantageously braked shortly before maximum extension is reached, in order to avoid a hard impact. A flexion movement is braked or prevented in the stance phase and in the swing phase, in order to ensure limitation of the bending.

Furthermore, a control device 60 and at least one angle detection device 70 are arranged on the orthopedic joint device. The angle detection device 70 detects the angle between the upper part 100 and the lower part 200 and is designed, for example, as a direct angle sensor, which detects the angle directly. Alternatively, the angle between the upper part 100 and the lower part 200 can be determined by evaluation of the sensor data of the spatial position sensors 50. Both methods can also be used simultaneously or to complement each other. All sensors arranged on the orthopedic joint device are coupled to a control device 60 and serve as a basis for controlling the actuator 1. On the basis of the sensor data, in particular the spatial positions and/or the angular positions and also position data and data on the deformation of further components, the actuator 1 is actuated, for example in order to reduce or increase a pivoting resistance, to limit an end stop, and/or to support a relative movement between the upper part 100 and the lower part 200.

FIG. 1 shows a schematic sectional representation of the actuator 1 in the form of a hydraulic actuator 1 with a main body 10, in which the cylinder 11 is formed or arranged. Located in the cylinder 11 is the piston 12, which is guided longitudinally displaceably along the longitudinal extent of the piston rod 20 in the cylinder 11. The piston 12 divides the cylinder 11 into two chambers 13, 14, which are fluidically connected to each other via an overflow channel 15. Arranged within the overflow channel 15 is a control valve or an adjustable throttle device 30, via which the flow resistance is adjustable. The throttle device 30 can be coupled to the electronic control device 60 and to an actuation element, wherein the control device 60 is coupled or can be coupled to sensors 16, 45, 50, 70 (not shown in FIG. 1) for detecting variables, parameters or other measured variables or measured values. On the basis of an evaluation of the measured values transmitted by the sensors, the control device 60 outputs a corresponding control command for activating, modulating or deactivating the actuation device and thus for adjusting the throttle device 30. Depending on the position of the throttle device 30, the flow resistance within the overflow channel 15 is increased or decreased, such that a reduced or increased resistance can be set against a movement of the piston 12 within the cylinder 11.

To be able to use the hydraulic damper 1, a first fastening device 21 is arranged or formed on the piston rod 20, via which the piston rod 20 can be secured to a displaceable component of a larger system, in particular an orthopedic joint device. For example, the fastening device 21 of the piston rod 20 can be secured to an upper part 100 or a lower part 200 of a prosthetic joint or orthotic joint, as is shown in FIGS. 8 and 9. A receiving device 40 is secured to the main body 10, at the end opposite the piston rod 20 and the fastening device 21 of the piston rod 20, and is connected to the main body 10. The main body 10 forms the housing for the cylinder 11, and the receiving device 40 is an extension or a continuation of the main body 10 on the side opposite the piston rod 20. Both the main body 10 and the receiving device 40 have a defined external dimension in the unloaded state.

On the side of the receiving device 40 opposite the piston rod 20 there protrudes a second fastening device 41 of the hydraulic actuator 1. The second fastening device 41 likewise serves to fasten the hydraulic actuator 1 to a second component, movable relative to the first component, of a larger system, for example an orthopedic joint device. If the first fastening device 21 is secured to the lower part 200 of a prosthetic or orthotic joint, the opposite second fastening device 41 of the receiving device 40 is secured to the upper part 100, and vice versa.

The fastening device 41 is mounted movably and elastically on the main body 10. This can be achieved, for example, by the receiving device 40 being formed as a spring, an elastomer element or another elastically resilient receiving device 40. Alternatively, the receiving device 40 is formed as a rigid housing in which the fastening device 41, designed for example as a tab with a hole or another bearing receptacle, is mounted movably and elastically. In the illustrated embodiment, the receiving device 40 is designed as a rigid housing which, in the installed state, is not displaceable relative to the main body 10.

If the hydraulic actuator 1 is subjected to a compressive force with which the piston rod 20 is pushed into the cylinder 11, then, in addition to the movement of the piston 12, there is a movement of the second fastening device 41 into the receiving device 40 or toward the piston 12. In a reverse application of force, for example during an extension, the first fastening device 21 and the second fastening device 41 are both moved away from each other, the piston rod 20 is pulled out of the main body 10, and likewise the fastening device 41 from the receiving device 40.

As an alternative to the illustrated passive embodiment of the hydraulic actuator 1, the latter can also be connected to a force accumulator or a pump so that, by suitable pressurization of the hydraulic fluid and feeding into one of the two chambers 13, 14, a corresponding displacement of the piston 12 in the direction of the lower pressure is effected. Instead of a hydraulic actuator, other actuator technologies are also conceivable, in particular electromechanical drives or braking and locking mechanisms. In principle, instead of being fastened to the main body 10, the receiving device 40 with associated fastening device 41 can also be fastened to the piston rod 20.

FIG. 2 shows the basic modular structure of the actuator 1 with the main body 10 and the first fastening device 21, e.g. on the piston rod, as an independent basic module via which the damping, braking and/or drive takes place. At the free end of the main body 10 provided with corresponding fastening means, different receiving means 40 can then be arranged interchangeably with the fastening devices 41. The fastening means are, for example, threads on the main body 10 and the receiving device 40, bayonet locks, screw devices or other form-fitting coupling elements. In the left-hand receiving device 40, the second fastening device 41 is supported displaceably and elastically within the receiving device 40; in the right-hand illustration, a variant of the module component is shown with a position sensor 45 via which it is possible to detect the respective position of the fastening device 41 relative to the main body 10 or to the receiving device 40. Accordingly, a position sensor 16 can also be arranged on the main body 10, via which position sensor the position of the fastening device 21 relative to the main body 10 can be detected. The position sensors 45, 16 serve to determine the length between the fastening devices 21, 41, which is variable depending on the height and direction of the load. From the displacement of the second fastening device 41 relative to the receiving device 40, it is possible to determine the force exerted on the actuator 1 and to derive the direction of force based on the knowledge of the spring stiffness and the course of the spring characteristic curve of the elastic mounting of the fastening device 41. On the basis of these values, it is possible for the control device 60 (not shown in FIG. 2) to activate or deactivate the actuator 1 or change resistances or supports. The position sensor 16, 45 can be designed, for example, as a magnetic, capacitive or inductive sensor; an optical sensor can also be used.

FIG. 2a shows the corresponding application of the actuator 1 in a rotational structure. The main body 10 of the rotary actuator 1 is connected to the lower part 200 via a fastening device 21. The receiving device 40 with rotational spring elements 43, 44 (not shown) is connected to the upper part 110 via the fastening device 41. Optionally, a position sensor 45 can be provided for detecting the position, in particular proportional to the deflection of the spring elements 43, 44, of the fastening device 41 relative to the receiving device 40. The receiving device 40 can, if necessary, be exchangeable and thus modular.

FIG. 3 shows a schematic sectional view of the mounting of the second fastening device 41 within the receiving device 40. In the illustrated embodiment, the receiving device 40 is designed as a rigid housing, in which the fastening device 41 is formed as a tab or rod with a bore, via which the receiving device 40 and thus also the entire hydraulic actuator 1 can be secured to an artificial joint, for example. The fastening device 41 protrudes into a cavity within the receiving device 40 and widens there in a piston-like manner and is supported by two spring elements 43, 44 relative to the receiving device 40. By spring elements 43, 44 being arranged on both sides and and being designed and oriented to counteract each other, the fastening device 41 is held in a defined starting position. A deflection of the spring elements 43, 44 is possible in both directions, so that the fastening device 41 is resiliently supported in both a retracting direction and an extending direction. In one embodiment, the spring elements 43, 44 have a non-linear, in particular progressive spring characteristic curve such that, in the event of increasing displacement in the direction of an end stop, there is an increased resistance to the further displacement. This avoids hard stops at the respective end position. The spring elements 43, 44 can be designed as helical springs, spiral springs, disk springs, disk spring packs and/or elastomer elements. Leaf springs can also be used as spring elements 43, 44. If the spring elements 43, 44 are provided with a pretensioning device 46, the pretensioning force can be adjusted, so that it is only from a predetermined load that a displacement of the fastening device 41 relative to the main body 10 takes place. In the case of non-linear springs, the pretensioning device 46 can also be used to manipulate the stiffness.

The respective position of the fastening device 41 within the receiving device 40 is detected via a position sensor 45. Knowledge of the spring behavior of the spring elements 43, 44, and of their position on the basis of the sensor data of the position sensor 45, allows a comparatively precise measured value to be obtained for the force effective within the displacement direction in or on the actuator 1. Moreover, these position data of the position sensor 45 can be transmitted to the control device 60 and serve to control the actuator 1.

In the illustrated embodiment, a pretensioning device 46 is arranged on the receiving device 40 and is assigned to a spring element 44. The pretensioning device 46 can be designed, for example, as a screw-in and screw-out abutment for the spring element 44 and serves to vary the pretensioning of the spring element 44. Such a pretensioning device 46 can also be assigned to the opposite spring element 43.

FIG. 3a shows an embodiment of FIG. 3 in which, alternatively or in addition to the position sensor 45, there is a blocking device 17 for maintaining the spring pretensioning.

Embodiments of the spring characteristic curves are shown in FIG. 4. The force-travel profile can be chosen to be arbitrarily non-linear, in particular progressive, as shown in the lower curve, wherein, on the basis of the known force-travel relationship, the force can be determined from the displacement and, in conjunction with a detected joint angle, the joint torque or the hydraulic force within the hydraulic actuator 1 can be determined. This value can be used for controlling the actuator 1, for activating a drive, for example for driving a pump for a hydraulic drive or for controlling the throttle device 30 of a hydraulic damper.

A variant of the invention is shown in FIG. 5, which largely corresponds to FIGS. 1 and 2. In addition to the elastic mounting of the fastening device 41, the piston 12 is assigned an auxiliary piston 121, which is mounted on the piston 12 via spring elements 441, which can be designed as a disk spring pack, for example. The position of the auxiliary piston 121 relative to the piston 12 is determined by the position sensor 16. It is also possible, as shown, for two position sensors 16 to be present, which respectively determine the position of the piston 12 and auxiliary piston 121 relative to a reference point or relative to each other.

By virtue of the serial arrangement of elastic elements or spring elements 43, 44, 441 within the hydraulic actuator 1, an elastic energy recovery is possible during the displacement of the upper part and lower part of a joint component or a relative displacement of components that are coupled to the hydraulic actuator. For example, in an application in an orthotic or prosthetic knee joint, the dynamics of the movement for the swing phase extension can be increased during the swing phase. Orthosis users also sometimes experience the problem that a complete swing phase extension cannot be performed, which leads to a foot being set down with the leg still bent. This results in increased energy expenditure in the stance phase, which can be reduced by extension support in the swing phase by converting stored potential energy in the spring elements.

In FIG. 5, the second fastening arrangement 41 and the receiving device 40 are designed as module components and can be interchangeably and permanently mounted on the main body 10 of the actuator 1 and secured thereon. The fastening device 41 is designed serially elastically relative to the main body 10, so that the entire actuator 1 can be adapted to different requirements by exchange or manipulation on the serially elastic module of the receiving device 40.

Advantageously, the total length in the unloaded state does not change when the individual module components are exchanged, so that the starting length of the actuator 1 results from the starting length 11 in the unloaded state of the main body and the starting length 12 of the fastening device 41 or the receiving device 40. During loading, the two fastening devices 21, 41 are displaced relative to each other, in particular moved in the longitudinal direction, such that the total length between the fastening devices 21, 42 can be increased or reduced relative to an unloaded starting position.

The stiffness of the spring elements 43, 44 (not shown in FIG. 5), which can be seen for example in FIG. 3, can be chosen to be different, such that a different resistance is provided under a load pushing together than under a load pulling apart.

The maximum displacement of the fastening devices 21, 41 relative to each other, and in particular the maximum displacement of the fastening device 41, can be limited by stops or by a progressive spring characteristic curve. As an alternative to two spring elements 43, 44, an elastic support can also be achieved using a spring or spring arrangement acting in both directions. In particular when using only one spring or a spring assembly with a plurality of spring components, these can be pretensioned such that a lower force threshold must be exceeded in order to achieve a change or displacement of the fastening device 41 relative to the main body 10.

Several springs or spring elements 43, 44 can be arranged in series or in parallel to one another. In addition to the spring elements 43, 44, the fastening device 41 can perform a damped movement. For this purpose, the fastening device 41 is assigned damping elements which have a dissipative effect in the event of a change in length. This can be done, for example, through a throttle opening or the arrangement of a solid-state damper.

In an arrangement, as shown in FIGS. 8 and 9, of the actuator 1 in an orthopedic joint device having an upper part and having a lower part mounted pivotably about a pivot axis, the joint device is assigned an angle detection device, which is designed as an angle sensor or has at least two spatial position sensors, one of which is arranged on the upper part and another on the lower part. The use of a linear actuator in such a joint device has the advantage that the extension moment on the actuator can be determined with less uncertainty. In the case of an elastic element arranged parallel to the actuator, a part of the force applied to the actuator is always taken up in a non-static load case, for example in the stance phase extension, such that the force calculated via a parallel elastic element and its spring characteristic is always lower than the actual extension force. In the embodiment of the actuator with the serially elastic arrangement according to the invention, the parallel component is omitted. In addition, the extension force and/or bending force can be measured in any joint angle position, even with a deactivated actuator. This is particularly advantageous when complete extension of the joint is to be prevented, but the moment applied is nevertheless essential for the control. For example, in order to protect an intact limb in the case of an orthotic fitting, a full extension or flexion of the joint may not be desired, so as to avoid too great an angle of extension or too great a flexion.

FIGS. 6 and 7 show both knee angle curves and manipulated values for the actuator, in particular the resistance to be applied in the direction of flexion over the course of a gait cycle. Thin lines represent the manipulated value for the actuator, thick lines represent the knee angle. The dashed line represents the energy stored in the spring element. A first illustration with a dotted line shows a purely passive embodiment of an artificial knee joint, for example a prosthetic knee joint or an orthotic knee joint without a component elastically mounted therein or thereon, a second illustration with a solid line shows the corresponding curves with an elastic component and the supply of stored potential energy in the course of the gait cycle. After a heel strike, there is a stance phase flexion with bending of a knee until, at approximately 20% of a gait cycle, a fully extended knee joint is present. In the stance phase, the knee joint remains predominantly extended, until it changes to the swing phase flexion at the end of the stance phase or shortly before toe-off. At about 70% of the gait cycle, the maximum flexion angle is reached and a movement reversal takes place in which the foot or a foot part performs an extension movement relative to a thigh part. Almost complete extension of the knee joint is reached by the end of the step cycle. This is indicated by the thick characteristic curves. The thin lines show the setpoint value of the actuator resistance in the direction of flexion. During the stance phase, the flexion is still obstructed or blocked such that, after the first stance phase flexion, the flexion is blocked in the case of an unsprung knee joint.

At about 25%, the flexion is enabled in order to be able to initiate a swing phase flexion. In the case of a spring-supported knee joint, the enabling occurs later on in this example, namely at about 30% of the gait cycle. During most of the swing phase, the bending movement is freely possible. To limit the maximum flexion angle, the bending movement is braked, which starts at about 60% of the gait cycle and leads to a slowing down of the pivoting movement of the lower part. After the movement reversal in the swing phase, the bending movement remains braked or locked in order to permit a safe set-down and, after the heel strike, a slight bending for the stance phase flexion.

For the system not provided with a spring according to the dotted line, which is evident from FIG. 7 which shows an enlarged, detailed representation of the swing phase, there is an early increase in the knee joint angle and an extended plateau, whereas, in the case of an elastically supported actuator component indicated by the solid line, there is a delayed increase in the knee joint angle with a more rapid reversal of movement. The area under the curve of the spring-supported embodiment is smaller than the area under the curve in the purely passive embodiment; the recovered energy is indicated by the dashed line. The curve in the embodiment with the supporting spring is much more uniform than in the purely passive embodiment and is almost sinusoidal, which is usually perceived positively by the users. The speed of the swing phase extension is higher with an activated spring than with a purely passive configuration.

The elasticity additionally provided in the actuator reduces the requirements on the swing phase regulator or a swing phase controller with regard to the precision at the switching times and the switching positions, and so the control hardware can be simplified. The regulator can be an essentially angle-dependent activation of the spring. Functionality is not limited to mechatronic systems. Since the spring elements of the elastic support are activated in a manner purely dependent on position, the system can also be used in combination with purely mechanical joints, in particular knee joints and swing phase controls.

If an energy storage device is present in the orthopedic joint device or in the actuator 1, making it possible to store converted energy when slowing down the movement of the upper part 100 relative to the lower part 200, this is advantageously achieved by the resistance being increased when a dynamically predefined target angle is reached, be it the maximum bending angle or an angle in the extension direction, which is shortly before the maximum extension. On the one hand, the movement is slowed down gently and, on the other hand, the energy necessary for this is stored. This stored energy can be returned to the system for supporting the movement reversal, for example for extension support or for flexion support, for example by releasing a spring or by enabling a compressed pneumatic volume. The time, the angle and/or the profile of the resistance increase can be designed to be individually adaptable in particular for the user depending on gait parameters, such as the walking speed, and/or auto-adaptive, that is to say self-adaptable over several steps on the basis of an optimal criterion.

FIG. 10 shows a schematic illustration of a spring-damper system which increases the degree of damping α_{F} as a function of the flexion angle φ_{K}. Forces F act in opposite directions on two fastening devices in a spring-damper system and change the distance Xᵣₑₗ between the two fastening points. This takes place, for example, in the case of a flexion of an artificial knee joint with an intermediate actuator 1. With a decreasing knee angle and an increasing flexion angle φ_{K}, the degree of damping α_{F} is increased starting from a defined flexion angle φ_{K}. In the case of an extension, it is the other way around: with a decreasing flexion angle φ_{K} and an increasing knee angle, i.e. an increasing extension, the degree of damping α_{F} increases starting from a defined threshold value. The increase in the degree of damping α_{F} can in particular take place progressively. In the first phase A of flexion or extension there is a comparatively low degree of damping, whereas in a second phase B there is a high degree of damping α_{F}. In the first phase A, which is shown in the top left figure, a resistance of the actuator or of the resistance device, for example a hydraulic damper, is comparatively low; in the case of a hydraulic actuator, the valves are then opened, such that a lower hydraulic resistance counteracts the corresponding movement. In a second phase B, the flow resistance or the resistance to the corresponding movement is increased by suitable measures, such that the serially elastic element acts starting from this point, for example at a flexion with a flexion angle φ_{K} of 65°.

1. An actuator, comprising:
a main body; and
at least one fastening device configured for fastening the actuator to components of an orthopedic device, wherein the at least one fastening device is mounted movably and elastically on the main body.

2. The actuator as claimed in claim 1, wherein the actuator is designed as a passive linear actuator selected from the group consisting of a hydraulic damper, a locking mechanism, and a brake.

3. The actuator as claimed in claim 1, wherein the actuator is designed as an active linear actuator selected from the group consisting of a hydraulic drive, and a drive.

4. The actuator as claimed in claim 1 wherein the at least one fastening device is mounted in a receiving device, wherein the at least one fastening device, in at least one direction of movement, is supported on the receiving device by at least one spring element.

5. The actuator as claimed in claim 4, wherein the at least one spring element is assigned at least one sensor for measuring deformation.

6. The actuator as claimed in claim 4 wherein, the receiving device is fastened releasably to the main body.

7. The actuator as claimed in claim 4 wherein the at least one spring element is mounted exchangeably in the receiving device.

8. The actuator as claimed in claim 4 wherein the at least one spring element is assigned an adjustable pretensioning device.

9. The actuator as claimed in claim 4 wherein the at least one spring element has a non-linear, progressive spring characteristic curve.

10. The actuator as claimed in claim 1 wherein the at least one fastening device is mounted in a damped manner.

11. The actuator as claimed in claim 1 wherein the at least one fastening device is assigned at least one position sensor (45).

12. The actuator as claimed in claim 1 wherein the actuator is designed as a linear, hydraulic actuator, wherein a cylinder is arranged or formed in which a piston is guided on a piston rod protruding from the main body, wherein the piston divides the cylinder into two chambers which are connected to each other via at least one overflow channel, and wherein at least one adjustable throttle device is arranged in the at least one overflow channel and is configured for adjusting a flow resistance in the at least one overflow channel.

13. The actuator as claimed in claim 12, wherein the at least one fastening device is mounted movably and elastically on the piston rod.

14. The actuator as claimed in claim 12 wherein the at least one fastening device is mounted movably in at least one displacement direction of the piston.

15. The actuator as claimed in claim 12 wherein the piston is assigned at least one position sensor.

16. The actuator as claimed in claim 12 further comprising an auxiliary piston mounted elastically on the piston, wherein the piston and the auxiliary piston are assigned at least one position sensor for detecting positions of the piston and the auxiliary piston or for detecting positions of the piston and the auxiliary piston relative to each other.

17. The actuator as claimed in claim 1 further comprising a control device coupled to at least one sensor, wherein the control device is configured to control the actuator based on sensor data from the at least one sensor.

18. An orthopedic joint device, comprising:
an upper part; (100),
a lower part mounted pivotably on the upper part about a pivot axis;
an actuator as claimed in claim 1 wherein the actuator is fastened to the upper part and to the lower part and provides resistance to a pivoting movement; and
an angle detection device assigned to the joint device.

19. The orthopedic joint device as claimed in claim 18, wherein the angle detection device is designed as an angle sensor, or the angle detection device has at least two spatial position sensors (50), one of which is arranged on the upper part and another of which is arranged on the lower part.

20. The orthopedic joint device as claimed in claim 18 wherein the angle detection device has a position sensor for detecting the actuator position and/or a position of the at least one fastening device.

21. A method for controlling an orthopedic joint device having an upper part, a lower part mounted pivotably on the upper part about a pivot axis (120), and an actuator arranged between the upper part and the lower part, wherein the actuator has a main body and at least one fastening device for fastening the actuator to the upper part or the lower part, wherein the actuator provides resistance to a pivoting movement of one or more of the upper part and the lower part, and wherein the resistance is changed as a function of sensor values, wherein a component of the actuator (1) is mounted elastically in or on the main body, comprising:
detecting position data of a component of the orthopedic device; and
changing the resistance of the actuator on the basis of the position data detected in the detecting step.

22. The method as claimed in claim 21, wherein the component for which the position data is detected is an elastically mounted component.

23. The method as claimed in claim 21 wherein the detecting step comprises detecting an angle between the upper part and the lower part. (200) is detected and the

24. The method as claimed in claim 23 wherein an angle sensor and/or a position sensor are/is used for detecting the position data of the component.

25. The method as claimed in claim 21 wherein the component and/or the actuator are/is assigned a blocking and enabling device which is moved from a blocking position to an enabling position, or vice versa, depending on sensor data.

26. The method as claimed in claim 21 further comprising increasing a resistance, using the actuator, when a dynamically predefined target angle is reached to permit elastic deceleration of the movement.

27. The method as claimed in claim 26, further comprising storing energy is during the elastic deceleration, and returning stored energy in order to support a reversal of movement.
